(19) United States
(12) Patent Application Publication
(10) Pub. No.: US 2025/0211845 A1
(43) Pub. Date: Jun. 26, 2025

(54) ELECTRONIC DEVICE, ENDOSCOPE, AND MANUFACTURING METHOD OF ELECTRONIC DEVICE

(71) Applicant: OLYMPUS MEDICAL SYSTEMS CORP., Tokyo (JP)

(72) Inventors: Yo ASAKURA, Tokyo (JP); Junya YAMADA, Tokyo (JP)

(73) Assignee: OLYMPUS MEDICAL SYSTEMS CORP., Tokyo (JP)

(21) Appl. No.: 19/073,612

(22) Filed: Mar. 7, 2025

Related U.S. Application Data
(63) Continuation of application No. PCT/JP2022/033914, filed on Sep. 9, 2022.

(51) Int. Cl. H04N 23/57; A61B 1/00; A61B 1/05; H04N 23/50; H10F 39/00

(52) U.S. Cl. CPC H04N 23/57; A61B 1/0011; A61B 1/05; H04N 23/555; H10F 39/811

(57) ABSTRACT

An electronic device includes: a circuit device having a specific region including two conductors formed on a surface of the circuit device to be spaced apart from each other by a first distance in a first direction and to have substantially equal widths in the first direction, and at least two solderless portions arranged to be spaced apart from each other by a second distance in a second direction intersecting the first direction and to intersect the two conductors; and an electronic component including two electrodes soldered to the two conductors, respectively, between the at least two solderless portions.

### CROSS-REFERENCE TO RELATED APPLICATION

This application is a continuation application of PCT/JP2022/033914 filed on Sep. 9, 2022, the entire contents of which are incorporated herein by this reference.

### FIELD OF DISCLOSURE

The present disclosure relates to an electronic device including a three-dimensional circuit device to which electronic components are mounted, an endoscope including the electronic device including the three-dimensional circuit device to which the electronic components are mounted, and a manufacturing method of the electronic device including the three-dimensional circuit device to which the electronic components are mounted.

### BACKGROUND

In recent years, three-dimensional circuit devices, for example, molded interconnect devices (MID) have been used for miniaturization and high functionality of electronic devices.

Japanese Patent Application Laid-Open Publication No. 2017-23234 discloses a camera unit of an endoscope. The camera unit includes an irregularly-shaped circuit substrate which is a three-dimensional circuit device. The camera unit includes an imager, a flat wiring board on which electronic components are mounted, and the irregularly-shaped circuit substrate. A cable is bonded to a pad of the irregularly-shaped circuit substrate.

In order to solder-bond a chip-type electronic component to a pad of a wiring board, a solder resist pattern configured to cover around the pad is placed for preventing short circuit between electrodes and preventing solder from flowing out.

WO Publication No. 2009/090896 discloses an electronic device in which an elongated insulating film (solder resist pattern) is placed on a conductor pattern formed on a principal surface of a flat wiring board.

The resist pattern is placed by patterning a spin-coated resist or a film resist using a photolithography method. The resist pattern is sometimes placed by screen-printing of the resist.

### SUMMARY

An electronic device according to one aspect of the present disclosure includes: a circuit device having a specific region including two conductors formed on a surface of the circuit device to be spaced apart from each other by a first distance in a first direction and to have substantially equal widths in the first direction, and at least two solderless portions arranged to be spaced apart from each other by a second distance in a second direction intersecting the first direction and to intersect the two conductors; and an electronic component including two electrodes soldered to the two conductors, respectively, between the at least two solderless portions.

An endoscope according to one aspect of the present disclosure includes: an insertion portion configured to be inserted into a subject; and an electronic device, the electronic device being disposed in the insertion portion. The electronic device includes: a circuit device having a specific region including two conductors formed on a surface of the circuit device to be spaced apart from each other by a first distance in a first direction and to have substantially equal widths in the first direction, and at least two solderless portions arranged to be spaced apart from each other by a second distance in a second direction intersecting the first direction and to intersect the two conductors; an electronic component including two electrodes soldered to the two conductors, respectively, between the at least two solderless portions; and a camera unit mounted on the circuit device.

A manufacturing method for an electronic device according to one aspect of the present disclosure includes: fabricating a circuit device by forming two conductors in a specific region on a surface of the circuit device, the two conductors being arranged to be spaced apart from each other by a first distance in a first direction and to have substantially equal widths in the first direction, and forming at least two solderless portions in the specific region, the at least two solderless portions being arranged to be spaced apart from each other by a second distance in a second direction intersecting the first direction and to intersect the two conductors; and soldering two electrodes of an electronic component to the two conductors, respectively, between the at least two solderless portions formed in the specific region of the circuit device.

### BRIEF DESCRIPTION OF THE DRAWINGS

FIG. 1 is a perspective view of an electronic device in a first embodiment.

FIG. 2 is a perspective view of the electronic device in the first embodiment.

FIG. 3 is a sectional view taken along the line III-III in FIG. 1.

FIG. 4 is a partial enlarged view of the electronic device in the first embodiment.

FIG. 5 is a flowchart of a manufacturing method of the electronic device in the first embodiment.

FIG. 6 is a partial enlarged view of an MID of the electronic device in the first embodiment.

FIG. 7A is a partial enlarged view for describing the manufacturing method of the electronic device in the first embodiment.

FIG. 7B is a partial enlarged view describing the manufacturing method of the electronic device in the first embodiment.

FIG. 8 is a partial enlarged view of the electronic device in the first embodiment.

FIG. 9 is a partial enlarged view of an electronic device in a modified example of the first embodiment.

FIG. 10 is a partial enlarged view of an electronic device in a modified example of the first embodiment.

FIG. 11 is a partial enlarged view of an electronic device in a modified example of the first embodiment.

FIG. 12 is a perspective view of an endoscope in a second embodiment.

### DETAILED DESCRIPTION

Hereinafter, embodiments of the present disclosure will be described with reference to drawings.

Note that the drawings based on each embodiment are schematic. The relationship between thicknesses and widths of respective parts, a ratio of a thickness of a certain part to that of another part, and the like of the respective parts are different from the actual ones. The respective drawings include parts in which the relationships and ratios among the dimensions are different.

### First Embodiment

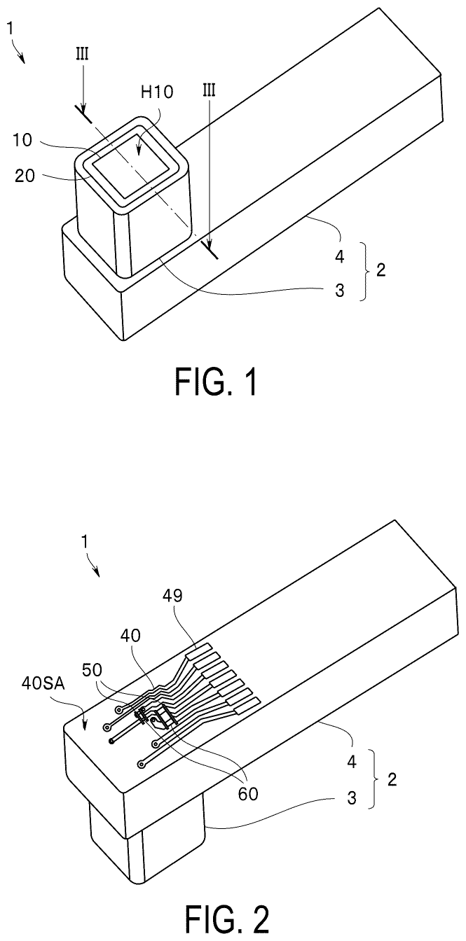

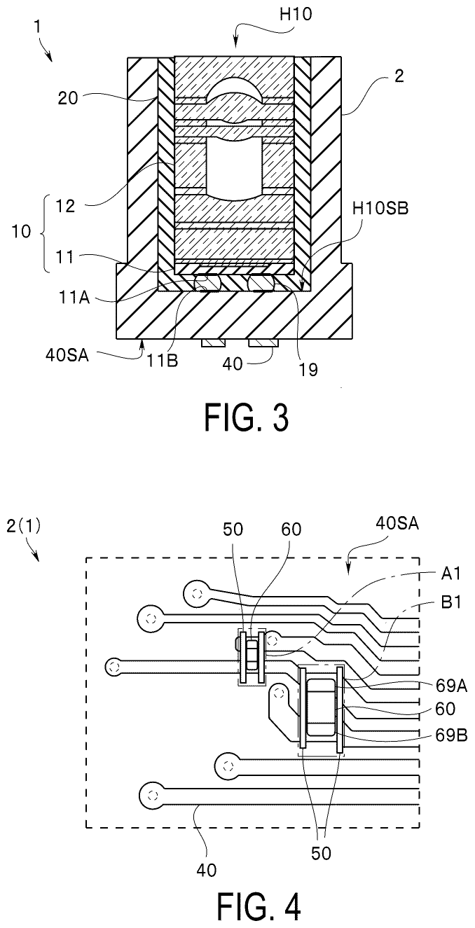

An electronic device in the present embodiment is shown in FIGS. 1 to 4. The electronic device in the present embodiment is an image pickup apparatus 1 that includes a three-dimensional circuit device 2 which is an MID (Molded Interconnect Device), a plurality of resist patterns 50, chip capacitors 60 (61, 62) as electronic components, and a camera unit 10.

The three-dimensional circuit device 2 is a three-dimensional molded interconnect device formed by placing a wiring on a surface of an injection-molded three-dimensional article. Unlike the conventional flat wiring board, using the three-dimensional circuit device 2 enables the shape thereof to have a function, and enables wiring to be formed on an inclined surface, a vertical surface, a curved surface, and in a through hole, etc.

The three-dimensional circuit device 2 has a complicated three-dimensional structure. Specifically, the three-dimensional circuit device 2 includes a protruded region 3 and an assembling region 4. The protruded region 3 configures a cavity H10 which is a hole in which the camera unit 10 is housed.

A gap between the camera unit 10 inside the cavity H10 and a wall surface of the cavity H10 is filled with a sealing resin 20. The camera unit 10 includes an imager 11 and an optical unit 12. The optical unit 12 includes a plurality of lenses, and the like. The imager 11 is an image sensor such as a CCD or the like that converts an object image, which is condensed by the optical unit 12, into an electric signal. The imager 11 includes an external electrode 11A that transmits and receives the electric signal.

A pad 11B on a bottom surface H10SB of the cavity H10 of the three-dimensional circuit device 2 is bonded to the external electrode 11A of the camera unit 10 using solder balls 19, for example. The pad 11B is connected to a land of a conductor pattern 40 on a surface 40SA, via a wiring (not shown). The surface 40SA is opposite to the bottom surface H10SB. The conductor pattern 40 includes, for example, a pad 49 that is bonded to a signal cable (not shown).

The camera unit 10 includes a by-pass capacitor for voltage stabilization. In the camera unit 10 which is driven at a high frequency, the by-pass capacitor may be connected to near the camera unit 10 in order to reduce an impedance of the wiring.

In the camera unit 10, the chip capacitors 60 (61, 62), which are chip electronic components, are surface-mounted on the surface 40SA of the three-dimensional circuit device 2. The chip capacitors 60 include two electrodes 69A and 69B. The chip capacitor 61 has a 0402 size (length is 0.4 mm, width is 0.2 mm). The chip capacitor 62 has a 0603 size (length is 0.6 mm, width is 0.3 mm).

In order to prevent the solder from spreading, two elongated solder resist patterns 50 (hereinafter, referred to as “resist patterns 50”) are placed. The solder resist patterns 50 cover around the pad as a bonding region between the chip capacitors 60 and the conductor patterns 40. In other words, the region of each of the conductor patterns 40, which is sandwiched by the two resist patterns 50, is the pad as the bonding region.

Hereinafter, the region in which the chip capacitor 61 is mounted is referred to as a first region A1. The region in which the chip capacitor 62 is mounted is referred to as a second region A2. Hereinafter, description will be made on the first region A1.

In the first region A1, two conductor patterns 41, 42 are arranged in parallel with each other with a predetermined first spacing. The widths of the conductor patterns 41, 42 are the same.

In the first region A1, the two resist patterns 50 are arranged in parallel with each other with a predetermined second spacing, and orthogonal to the two conductor patterns 40. With the two resist patterns 50, a part of each of the conductor patterns 41 and 42 become each of the two pads to which the two electrodes 69A and 69B of the chip capacitor 61 are solder-bonded. The electrode 69A is bonded to the pad of the conductor pattern 41. The electrode 69B is bonded to the pad of the conductor pattern 42.

Here, it is assumed that the widths W40 of the two conductor patterns 41 and 42 are the same. However, such two conductor patterns 41 and 42 include the ones that have substantially the same width with a slight error (for example, 90% to 110% of the average value of the both). It is assumed that the two resist patterns 51 and 52 are in parallel with each other. However, such two resist patterns 51 and 52 include the ones which are not completely parallel with each other but substantially parallel with each other with a slight error (for example, the intersecting angle is smaller than 10 degrees). It is assumed that the two resist patterns 50 are orthogonal to the two conductor patterns 40. However, such two resist patterns include the ones which are not completely orthogonal (the intersecting angle is 90 degrees) but substantially orthogonal to the two conductor patterns with a slight error (for example, the intersecting angle is 80 to 100 degrees). Since the respective components have very small sizes and are manufactured by performing minute processing, errors are likely to occur. Therefore, the components are configured as described above in the present disclosure.

As will be described later, the two pads for bonding the chip capacitor 61 having a predetermined size are required to have a size, a spacing, and the like that are set in predetermined ranges. In other words, the two conductor patterns 40 are designed to configure the two pads for bonding the chip capacitor 61 in the first region A1.

It is not easy to place the resist patterns on the surface 40SA of the three-dimensional circuit device 2 which has the complicated shape. In other words, unlike the flat wiring board, it is difficult to apply spin coating or screen printing to the surface of the three-dimensional circuit device. In addition, it is also difficult to form the resist by patterning on the surface of the three-dimensional circuit device using the photolithography method. Furthermore, the surface 40SA of the three-dimensional circuit device 2 is extremely small, i.e., the size of which is smaller than 100 mm² (for example, 3 mm×2.5 mm). It is difficult to surface-mount electronic components on the three-dimensional circuit device 2.

However, as described later, the resist patterns 50 are placed by using a dispenser. Therefore, each of the resist patterns 50 is formed in an elongated bank-like shape, the height of which is 25 μm, for example. The expression “elongated” refers to a shape having a length more than five times a width, for example. The conductor patterns 41 and 42 do not have to be parallel with each other in the entire range of the first region A1, as long as the conductor patterns are parallel with each other in the region located between the resist patterns 51 and 52.

When solder 69 is placed on the conductor patterns 41 and 42 before arranging the chip capacitor 61, there is a case where the solder 69 is applied also onto the resist patterns 50. Each of the resist patterns 50 has a poor solder wettability (poor easiness of affinity with the solder). For this reason, the solder 69 on the resist patterns 50 is repelled in the reflow soldering, to move onto the conductor patterns 40. The solder 69 wet-spreads only onto the conductor patterns 41 and 42, which forms a solder-non-wetting area to which the solder 69 does not adhere on the conductor patterns 40 in the first region. The resist patterns 50 are the solder-non-wetting areas.

The solder wettability is measured, for example, by “solder bath wetting balance method” specified by the Japanese Industrial Standards Z3198-4. A region with a long zero cross time (time taken for the contact angle to become 90 degrees after immersion) has a poorer solder wettability than a region with a short zero cross time.

When the image pickup apparatus 1 is in the state of a finished product, it is impossible to directly identify that the resist patterns 50 have been placed by using the dispenser.

The image pickup apparatus 1 has a high performance, since the chip capacitors 60 are placed on the surface 40SA of the three-dimensional circuit device 2 and the wiring between the surface 40SA and the camera unit 10 is short.

Note that, in the image pickup apparatus 1, the two chip capacitors 61 and 62 having different sizes are mounted on the same surface 40SA. However, the electronic components may be chip inductors or chip coils, for example, as long as the electronic components each have two electrodes. The electronic component including three or more electrodes is not mounted to the first region A1. Three kinds or more electronic components each having two electrodes may be mounted to the first region A1. A plurality of electronic components may be mounted on a surface other than the first region A1 of the three-dimensional circuit device 2.

### Manufacturing Method of the Electronic Device

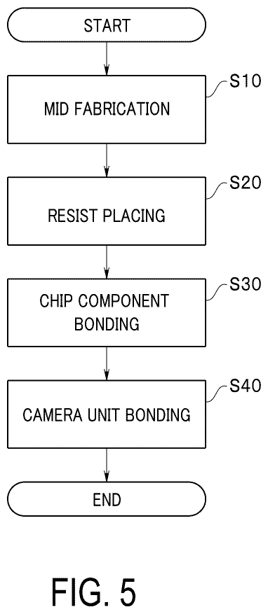

The manufacturing method of the image pickup apparatus 1 will be described with reference to the flowchart in FIG. 5.

### <Step S10>Three-Dimensional Circuit Device Fabrication Step

A molded body is fabricated by injection molding performed by injecting an MID resin into a mold (not shown) including the shape of the three-dimensional circuit device 2. The surface of the molded body is irradiated with laser, to thereby form a region having a catalytic activity of electroless plating. Furthermore, wirings (not shown), which connect the pad 11B on the bottom surface H10SB of the cavity H10 and the conductor patterns 40, are formed. After that, electroless plating processing is performed, and the molded body becomes the three-dimensional circuit device (three-dimensional circuit device 2) including on the surface thereof a plurality of conductor patterns 40 placed. Each of the conductor patterns 40 includes, for example, on the copper layer thereof, a nickel plating film, and includes on the outermost surface thereof, a gold plating film.

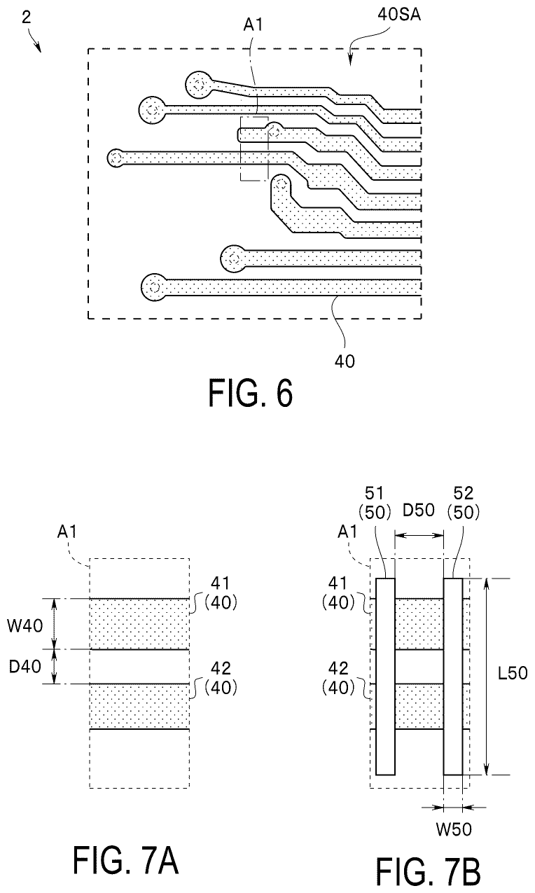

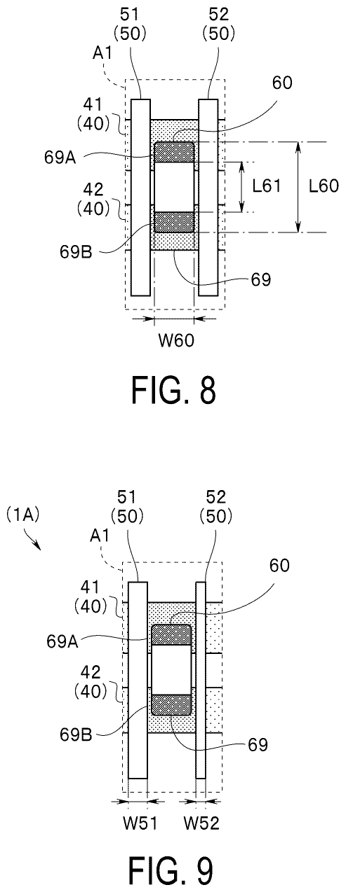

As shown in FIG. 6, the plurality of conductor patterns 40 are placed on the surface 40SA of the three-dimensional circuit device 2. The region where, among the plurality of conductor patterns 40, the two conductor patterns 41 and 42 having substantially the same width are arranged in parallel with each other is referred to as the first region A1. FIG. 7A shows the first region A1 shown in FIG. 6. As shown in FIG. 7A, the conductor patterns 41 and 42 are arranged in parallel with each other with a first spacing D40 therebetween. The widths W40 of the conductor patterns 41 and 42 are the same.

### <Step S20>Resist Placing Step (Solder-Non-Wetting Area Placing Step

As shown in FIG. 7B, the two elongated resist patterns 50 (51, 52) are placed in the first region A1 by using a dispenser. The two resist patterns are arranged in parallel with each other with a second spacing D50 therebetween. In the present embodiment, the width W50 of the resist pattern 51 is the same as the width W50 of the resist pattern 52, and the length L50 of the resist pattern 51 is the same as the length L50 of the resist pattern 52.

The length L50 of each of the resist patterns 50 is longer than the length L60 (0.4 mm in the case of the 0402 size) of each of the chip capacitors 60 to be mounted.

Unlike the resist patterns patterned by the photolithography method, in the two resist patterns 50 placed by the dispenser, there is a case where the placing start position (the position of one end of each of the resist patterns 50) deviates from the desired position due to the positioning accuracy of the dispenser. In view of the positional deviation, the length L50 may be more than 1.2 times of the length L60. The upper limit of the length L50 is, for example, less than three times of the length L60.

In a case where each of the resist patterns 50 is formed in a frame shape, if the positional deviation occurs in the direction of the length L60, there is a case where the areas of the two pads are different. However, the areas of the two pads formed by the two elongated resist patterns 51 and 52 are substantially the same, even if the positional deviation in the direction of the length L60 occurs in the resist patterns 51 and 52, similarly as in the case where no positional deviation occurs.

### <Step S30>Chip Component Bonding Step

Although not shown, the solder 69 (69A, 69B) is placed, by using the dispenser, on each of the two conductor patterns 41 and 42 between the two resist patterns 51 and 52.

As shown in FIG. 9, the chip capacitor 60 is arranged at a predetermined position in the first region A1.

For example, the three-dimensional circuit device 2 is heated to a temperature at which the solder melts by using a reflow furnace. The temperature of the three-dimensional circuit device 2 returns to a room temperature, the two electrodes 69A and 69B of the chip capacitor 60 are solder-bonded.

Although the description has been omitted, the chip capacitor 62 is bonded also to the second region A2 at the same time as the chip capacitor 61.

### <Step S40>Camera Unit Bonding Step

For example, the camera unit 10 is fabricated by cutting a stacked wafer formed by adhering imager chips on a plurality of optical wafers. The solder ball (solder) 19 is placed on each of the plurality of external electrodes 11A of the camera unit 10. Instead of the solder ball 19, a solder plating film or a solder paste may be placed on each of the external electrodes 11A.

The camera unit 10 is housed in the cavity H10 of the three-dimensional circuit device 2. Then, the reflow furnace is used, for example, and the three-dimensional circuit device 2 is heated to a temperature at which the solder balls 19 melt. The solder balls 19 spread to the pads formed by the resist patterns 50. In other words, the widths of the pads that are solder-bonded to the electrodes are the width W40 of the conductor patterns 40, and the lengths of the pads are the spacing D50 of the two resist patterns 50. The shape of each of the pads is substantially rectangular, and each of the pads has an area of (W40×D50).

When the temperature of the three-dimensional circuit device 2 returns to the room temperature, the external electrodes 11A of the camera unit 10 are solder-bonded to the pad 11B on the bottom surface H10SB of the cavity H10, to thereby complete the image pickup apparatus 1.

For stable bonding, in the first region A1 in which the chip capacitor 61 of the 0402 size is placed, the first spacing D40 between the conductor patterns 41 and 42 may be 0.16 mm to 0.20 mm, and the widths W41 and W42 of the conductor patterns 40 may be 0.12 mm to 0.18 mm. The second spacing D50 between the resist patterns 51 and 52 may be more than 0.20 mm and less than 0.30 mm.

In the region B1 in which the chip capacitor 62 of the 0603 size is placed, the first spacing D40 between the conductor patterns 41 and 42 may be 0.20 mm to 0.30 mm, and the widths W40 of the conductor patterns 41 and 42 may be 0.20 mm to 0.35 mm. The second spacing D50 between the resist patterns 51 and 52 may be more than 0.30 mm and less than 0.40 mm.

As described above, the width W40 of each of the conductor patterns 40 may be more than 70% and less than 130% to the first spacing D40 between the conductor patterns 40, and the first spacing D40 between the conductor patterns 40 may be more than 100% and less than 180% to the second spacing D50 between the resist patterns 50. In addition, the second spacing D50 between the resist patterns 50 may be more than 0.20 mm and less than 0.40 mm.

The width W50 of each of the resist patterns 50 that are placed by using the dispenser is more than 0.10 mm and less than 0.5 mm.

It is not easy to place the resist patterns on the surface 40SA, which has a small area, of the three-dimensional circuit device 2 having the complicated shape. However, it is not difficult to place the two line-shaped resist patterns 50 by using the dispenser. In the present embodiment, the conductor patterns 40 between the two resist patterns 50 can be the pads to which the chip capacitors 60 are solder-bonded.

As already described above, the two pads defined by the two resist patterns 50 that are substantially parallel with each other and intersect with the two parallel conductor patterns 41 and 42 have the same area, even if a positional deviation occurs in the resist patterns 50. When the solder is melted, the solder 69 spreads to the two pads in the same manner. The interfacial tensions of the melted solder applied to the two electrodes 69A and 69B of the chip capacitor 60 are the same, which causes no occurrence of bonding defects such as the Manhattan phenomenon or a decrease in the bonding strength. The present manufacturing method enables the chip capacitor 60 to be easily solder-bonded to the three-dimensional circuit device 2.

### Modified Examples of First Embodiment

Image pickup apparatuses 1A to 1C in the modified examples of the first embodiment are similar to and have the same effects as the image pickup apparatus 1 in the first embodiment. Therefore, in the description below, the same constituent elements having the same functions as those of the image pickup apparatus 1 are attached with the same reference signs and descriptions thereof will be omitted.

### Modified Example 1

As shown in FIG. 9, in the image pickup apparatus 1A in the present modified example, a width W51 of a resist pattern 51 is different from a width W52 of a resist pattern 52 in a three-dimensional circuit device 2A. In other words, the widths W50 of the two resist patterns 50 may be different from each other. In addition, although not shown, the lengths L50 of the two resist patterns 50 may be different from each other.

### Modified Example 2

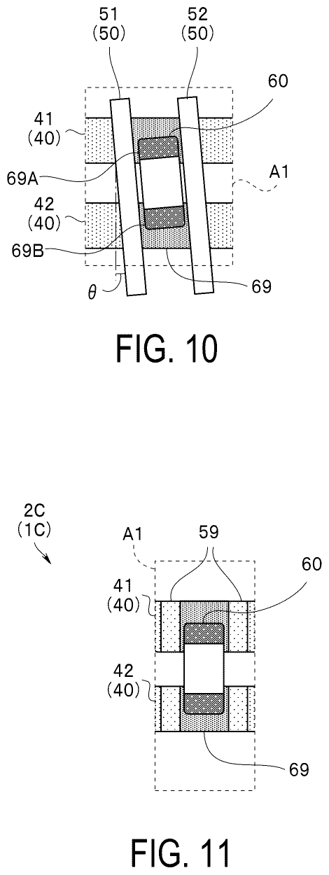

As shown in FIG. 10, in the image pickup apparatus 1B in the present modified example, resist patterns 50 incline at an angle θ with respect to conductor patterns 40 in a three-dimensional circuit device 2B.

In other words, the two resist patterns 50 may be substantially parallel with each other and orthogonal to the conductor patterns 40 (θ=0), but may be substantially inclined (for example, θ<10 degrees) with respect to the conductor patterns 40.

Note that, although not shown, the two conductor patterns 41 and 42 may be substantially parallel with each other. For example, an angle θ formed by the conductor pattern 41 and the conductor pattern 42 may be less than 10 degrees.

However, the two pads may have substantially the same shape and substantially the same area, for example, the area of one of the pads may be more than 90% and less than 110% to the area of the other of the pads.

### Modified Example 3

As shown in FIG. 11, in the image pickup apparatus 1C in the present modified example, a three-dimensional circuit device 2C is configured such that conductor patterns 40 in a first region A1 include four peeling-off regions 59 in which the solder wettability is poorer than in regions around these peeling-off regions 59. In the peeling-off regions 59, the gold plating films formed on the surfaces thereof are peeled off by using laser.

The peeling-off regions 59 with poor solder wettability have the same effects as the resist patterns 50. In other words, when the electronic components 60 are solder-bonded, the peeling-off regions 59 with poor solder wettability become the solder-non-wetting areas to which the solder 69 is not adhered.

The shape and the like of the peeling-off regions 59 having the poor solder wettability may be the same as the shape and the like of the resist patterns 50.

The image pickup apparatus 1C has a higher degree of freedom in manufacturing than the image pickup apparatus 1 and the like.

### Second Embodiment

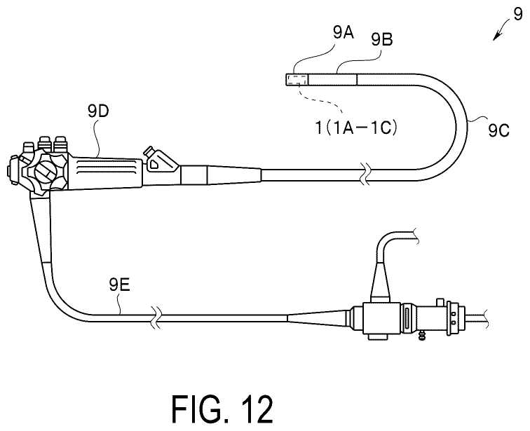

An endoscope 9 in the present embodiment shown in FIG. 12 includes a rigid distal end portion 9A in which the image pickup apparatus 1 (1A to 1C) is provided, a bendable bending portion 9B provided continuously with the proximal end of the rigid distal end portion 9A, and an elongated flexible portion 9C provided continuously with the proximal end of the bending portion 9B. The bending portion 9B is bent by operating an operation portion 9D. The rigid distal end portion 9A, the bending portion 9B, and the flexible portion 9C constitute an insertion portion configured to be inserted into a body of a subject. A universal cord 9E extended from the operation portion 9D is connected to a processor, etc., not shown. The endoscope 9 includes the image pickup apparatus 1 (1A to 1C), and thereby achieves a high performance.

Note that the endoscope 9 is a flexible endoscope for medical use. However, an endoscope in another embodiment may be an endoscope for industrial use, and may be a rigid endoscope having a rigid straight pipe, instead of the flexible portion 9C.

In addition, the three-dimensional circuit device is not limited to the MID. The three-dimensional circuit device may be fabricated by processing using a 3D-printer, or cutting processing, for example. The material of the three-dimensional circuit device is not limited to the resin, but ceramics or glass epoxy may be used as the material. In addition, the electronic device is not limited to the image pickup apparatus.

The present disclosure is not limited to the above-described embodiments, or the like, but various changes, modifications, etc., are possible without changing the gist of the present disclosure.
Example 1. An electronic device comprising:

a three-dimensional circuit device including a surface on which two conductor patterns are provided, the surface including a first region in which the two conductor patterns are arranged in parallel with each other with a first spacing and widths of the two conductor patterns are substantially same;

a plurality of solder-non-wetting areas to which solder is not adhered, the plurality of solder-non-wetting areas being arranged in the first region so as to be in substantially parallel with each other with a second spacing and substantially orthogonal to the two conductor patterns; and

at least one electronic component including two electrodes that are solder-bonded respectively to the two conductor patterns between the plurality of solder-non-wetting areas.
Example 2. The electronic device according to Example 1, wherein widths of the plurality of solder-non-wetting areas are substantially same.
Example 3. The electronic device according to Example 1, wherein widths of the plurality of solder-non-wetting areas are different from each other.
Example 4. The electronic device according to Example 1, wherein the two conductor patterns are substantially orthogonal to the plurality of solder-non-wetting areas.
Example 5. The electronic device according to Example 1, wherein

the widths of the two conductor patterns are more than 70% and less than 130% to the first spacing, and

the first spacing is more than 100% and less than 180% to the second spacing.
Example 6. The electronic device according to Example 1, wherein

the first spacing is more than 0.16 mm and less than 0.30 mm, the widths of the two conductor patterns are more than 0.12 mm and less than 0.35 mm, and

the second spacing is more than 0.20 mm and less than 0.40 mm.
Example 7. The electronic device according to Example 1, wherein the electronic component is a capacitor.
Example 8. The electronic device according to Example 1, wherein the three-dimensional circuit device is a molded interconnect device.
Example 9. The electronic device according to Example 1, wherein the plurality of solder-non-wetting areas are two elongated resist patterns.
Example 10. The electronic device according to Example 9, wherein the resist patterns are placed by using a dispenser.
Example 11. The electronic device according to Example 1, wherein

the two conductor patterns each include an outermost surface on which a gold plating film is placed, and

the plurality of solder-non-wetting areas are regions where the gold plating film of each of the two conductor patterns is peeled off.
Example 12. The electronic device according to Example 1, further comprising a camera unit mounted to the three-dimensional circuit device.
Example 13. The electronic device according to Example 1, further comprising a plurality of electronic components each having a different size.
Example 14. An endoscope comprising an insertion portion configured to be inserted into a subject and an electronic device provided in the insertion portion and including a camera unit,

the electronic device comprising:

a three-dimensional circuit device including a surface on which two conductor patterns are provided, the surface including a first region in which the two conductor patterns are arranged in parallel with each other with a first spacing and widths of the two conductor patterns are substantially same;

a plurality of solder-non-wetting areas to which solder is not adhered, the plurality of solder-non-wetting areas being arranged in the first region so as to be in substantially parallel with each other with a second spacing and substantially orthogonal to the two conductor patterns; and

an electronic component including two electrodes that are solder-bonded respectively to the two conductor patterns between the plurality of solder-non-wetting areas.
Example 15. A manufacturing method of an electronic device, the manufacturing method comprising:

fabricating a three-dimensional circuit device including a surface on which two conductor patterns are provided, the surface including a first region in which the two conductor patterns are arranged in parallel with each other with a first spacing and widths of the two conductor patterns are substantially same;

placing a plurality of solder-non-wetting areas in the first region so as to be in parallel with each other with a second spacing and substantially orthogonal to the two conductor patterns; and

solder-bonding two electrodes of a chip electronic component respectively to the two conductor patterns between the plurality of solder-non-wetting areas.

What is claimed is:

1. An electronic device comprising:
a circuit device having a specific region comprising:
two conductors formed on a surface of the circuit device to be spaced apart from each other by a first distance in a first direction and to have substantially equal widths in the first direction; and
at least two solderless portions arranged to be spaced apart from each other by a second distance in a second direction intersecting the first direction and to intersect the two conductors; and
an electronic component comprising two electrodes soldered to the two conductors, respectively, between the at least two solderless portions.

2. The electronic device according to claim 1, wherein
the circuit device is a three-dimensional circuit device,
the two conductors extend substantially parallel to each other in the second direction orthogonal to the first direction, and
the at least two solderless portions extend substantially parallel to each other in the first direction.

3. The electronic device according to claim 2, wherein
an angle between the two conductors is less than 10 degrees,
the widths of the two conductors are within a range of 90% to 110% of an average value of the widths of the two conductors, and
an angle between the at least two solderless portions is less than 10 degrees.

4. The electronic device according to claim 1, wherein the at least two solderless portions have substantially equal widths in the second direction.

5. The electronic device according to claim 1, wherein the at least two solderless portions have different widths in the second direction.

6. The electronic device according to claim 1, wherein
the widths of the two conductors are greater than 70% but less than 130% of the first distance, and
the first distance is greater than 100% but less than 180% of the second distance.

7. The electronic device according to claim 1, wherein
the first distance is greater than 0.16 mm but less than 0.30 mm,
the widths of the two conductors are greater than 0.12 mm but less than 0.35 mm, and
the second distance is greater than 0.20 mm but less than 0.40 mm.

8. The electronic device according to claim 1, wherein the electronic component comprises a capacitor.

9. The electronic device according to claim 1, wherein the circuit device is a molded interconnect device.

10. The electronic device according to claim 1, wherein the at least two solderless portions are two line-shaped resist portions.

11. The electronic device according to claim 1, wherein
each of the two conductors comprises a gold plating layer applied on an outer surface of each conductor, and
each of the at least two solderless portions has a region without the gold plating layer on the outer surface of a corresponding one of the two conductors.

12. The electronic device according to claim 1, further comprising a camera unit mounted on the circuit device.

13. The electronic device according to claim 12, wherein the circuit device comprises:
a first surface on which the camera unit is mounted; and
a second surface opposite to the first surface, the second surface having the specific region.

14. The electronic device according to claim 1, further comprising an additional electronic component,
wherein the electronic component and the additional electronic component have different sizes.

15. The electronic device according to claim 1, wherein each solderless portion extends across the two conductors from a first end to a second end in the first direction, the first end and the second end of each solderless portion being positioned outside the two conductors in the first direction.

16. The electronic device according to claim 1, wherein
the two conductors comprise:
a first conductor; and
a second conductor spaced apart from the first conductor by the first distance in the first direction,
the at least two solderless portions comprise:
a first solderless portion positioned on the first conductor;
a second solderless portion positioned on the first conductor and spaced apart from the first solderless portion by the second distance in the second direction;
a third solderless portion positioned on the second conductor;
a fourth solderless portion positioned on the second conductor and spaced apart from the third solderless portion by the second distance in the second direction, and
the two electrodes of the electronic component comprise:
a first electrode soldered to the first conductor between the first solderless portion and the second solderless portion; and
a second electrode soldered to the second conductor between the third solderless portion and the fourth solderless portion.

17. An endoscope comprising:
an insertion portion configured to be inserted into a subject; and
the electronic device according to claim 12, the electronic device being disposed in the insertion portion.

18. A manufacturing method for an electronic device, comprising:
fabricating a circuit device by:
forming two conductors in a specific region on a surface of the circuit device, the two conductors being arranged to be spaced apart from each other by a first distance in a first direction and to have substantially equal widths in the first direction; and
forming at least two solderless portions in the specific region, the at least two solderless portions being arranged to be spaced apart from each other by a second distance in a second direction intersecting the first direction and to intersect the two conductors; and
soldering two electrodes of an electronic component to the two conductors, respectively, between the at least two solderless portions formed in the specific region of the circuit device.

19. The manufacturing method according to claim 18, further comprising placing two resist portions using a dispenser, thereby forming the at least two solderless portions.

20. The manufacturing method according to claim 18, further comprising:
applying a gold plating layer on an outer surface of each of the two conductors; and
partially removing the gold plating layers on the two conductors, thereby forming the at least two solderless portions.
